(19) United States
(12) Patent Application Publication
(10) Pub. No.: US 2016/0109397 A1
(43) Pub. Date: Apr. 21, 2016

(54) REACTION MATERIAL CARTRIDGE FOR MEASURING OUTDOOR AIR PENETRATION RATE AND DEVICE FOR MEASURING OUTDOOR AIR PENETRATION RATE OF SUBSTRATE, INCLUDING SAME

(71) Applicant: KOREA ELECTRONICS TECHNOLOGY INSTITUTE, Seongnam-si, Gyeonggi-do (KR)

(72) Inventors: Won Keun KIM, Seongnam-si (KR); Chul Jong HAN, Yongin-si (KR); Soon Hyung KWON, Seongnam-si (KR); Jeong No LEE, Yongin-si (KR); Min Suk OH, Seoul (KR); Ji Wan KIM, Seongnam-si (KR); Byung Wook YOO, Seongnam-si (KR); Yong Hoon KIM, Seongnam-si (KR)

(73) Assignee: KOREA ELECTRONICS TECHNOLOGY INSTITUTE, Seongnam-si, Gyeonggi-do (KR)

(21) Appl. No.: 14/893,554

(22) PCT Filed: May 29, 2014
(86) PCT No.: PCT/KR2014/004802

§ 371 Date: Nov. 24, 2015

(30) Foreign Application Priority Data
May 30, 2013 (KR) 10-2013-0061787

(51) Int. Cl. G01N 27/20; G01N 33/00

(52) U.S. Cl. CPC G01N 27/205; G01N 33/0004; G01N 33/0027

(57) ABSTRACT

A device for measuring a penetration rate of outdoor air including humidity or oxygen of a material, and a reaction material cartridge for measuring an outdoor air penetration rate. The device includes a reacting part including a reaction material which reacts upon contact with outdoor air passing through a substrate; a cartridge body which accommodates the reaction part, and which is detachable from a measuring unit for measuring a change corresponding to the contact of the reaction material with the outdoor air; and a shielding means which shields the cartridge body, and which is separated from the cartridge body for contact between the reaction material and the outdoor air.

### TECHNICAL FIELD

This invention relates to a device and a method for measuring a penetration rate of outdoor air which includes humidity, oxygen, etc. into a material.

### BACKGROUND ART

Active matrix organic light emitting diodes (AMOLED) currently expected to be commercialized use various organic materials such as an electron-transport layer (ETL), an emissive layer (EML), a hole injection layer (HIL), a hole transport layer (HTL), etc. in technical properties. However, since organic materials described above are vulnerable to humidity which exists in the atmosphere, it is necessary to well protect a panel from humidity penetration thereinto to provide reliability of the panel.

Particularly, in the case of AMOLED, it has been known that it is possible to embody a reliable product when a humidity penetration rate or a water vapor transmission rate (WVTR) of a substrate satisfies 10⁻⁶ g/m²-day or less. However, in the case of AQUATRAN Modell of MOCON Inc., a measurement limit is about 5×10⁻⁴ g/m²-day which does not satisfy a WVTR reference of 10⁻⁶ g/m²-day substantially necessary for an AMOLED panel.

To overcome such limitations, Philips Electronics has provided a method of reversely tracing a WVTR using calcium which very quickly responds to humidity (disclosed in U.S. Pat. No. 7,117,720 titled “Method for measuring a permeation rate, a test and an apparatus for measuring and testing”) . The method called a “calcium test” is a technology in which a calcium electrode is formed on a substrate to be measured with a WVTR, an exposed rear surface portion is encapsulated with a metal or glass, and an amount of gas which flows through the substrate is extracted by analyzing transmittance of a calcium layer.

Also, Korea University Research & Business Foundation has provided a method of analyzing a permeation rate of gas which penetrates by tracing a change in an electrical conductivity or resistance instead of a transmittance of a calcium layer while being manufactured in a similar form to the described above (disclosed in Korean Patent Publication No. 10-2013-0022068 titled “Gas permeation rate measurement apparatus of flexible electronic device protection layer and gas permeation rate measurement method using the same”).

Theoretically, in the case of calcium, it is possible to measure a WVTR of 10⁻⁶ g/m²-day or less. However, since an additional vacuum deposition apparatus is necessary to deposit calcium and many process parameters occur in analyzing a WVTR depending on a calcium deposition method, a test sample structure, and a process method, as a result, great difficulties are present in providing reliable data. For this reason, it may be very difficult to generalize the currently known “calcium test” method as an existing WVTR analyzing technology of MOCON Inc.

### SUMMARY OF INVENTION

### Technical Problem

It is an aspect of the present invention to provide a method of measuring a penetration rate of outdoor air into a substrate by manufacturing a calcium electrode and components related to measurement of resistance, penetration degree, or the like as cartridges.

In more detail, it is another aspect of the present invention to provide a device which increases reliability and precision of measurement by forming key components necessary for precise measurement as cartridges.

### Solution to Problem

One aspect of the present invention provides a reactant cartridge for measuring an outdoor air penetration rate of a substrate, including a reacting portion which includes a reactant which reacts according to a contact with outdoor air which penetrates through the substrate, a cartridge body which accommodates the reacting portion and is detachable from a measuring unit which measures a change of the reactant according to the contact with the outdoor air, and a shielding means which shields the cartridge body and is detached from the cartridge body to allow the reactant to be in contact with the outdoor air.

The substrate may be accommodated in a substrate cartridge which includes a substrate cartridge body detachable from a supply pipe through which the outdoor air including humidity is supplied and substrate shielding means which shield the substrate cartridge body and are detached from the substrate cartridge body to allow the substrate to be in contact with the outdoor air or to allow the reactant to be in contact with the outdoor air which penetrates through the substrate.

Another aspect of the present invention provides a device for measuring an outdoor air penetration rate of a substrate which includes a measuring unit including an outdoor air environment control chamber which controls an environment of supplied outdoor air, a supply pipe which supplies the outdoor air controlled with the environment, a substrate mounting portion located in a first predetermined space of the supply pipe, on which the substrate to be measured with a penetration rate is mounted, and a cartridge mounting portion located in a second predetermined space of the supply pipe, on which a reactant cartridge which includes a reactant which reacts according to a contact with outdoor air which penetrates through the substrate is mounted, wherein the measuring unit measures a change of the reactant according to the contact with the outdoor air.

The substrate may be accommodated in a substrate cartridge which includes a substrate cartridge body detachable from the supply pipe through which the outdoor air including humidity is supplied and substrate shielding means which shield the substrate cartridge body and are detached from the substrate cartridge body to allow the substrate to be in contact with the outdoor air or to allow the reactant to be in contact with the outdoor air which penetrates through the substrate, and the substrate cartridge may be mounted on the substrate mounting portion.

### Advantageous Effects of Invention

According to the present invention, as a cartridge-based gas penetration rate measuring device and method are used, it is possible to provide precise and reliable data when compared to conventional techniques, and to facilitate product reliability assessments necessary for new application fields such as a flexible active matrix organic light emitting diode (AMOLED) and the like. In addition, a substrate accommodating portion is also manufactured in the form of a cartridge in such a way that when a flexible substrate which is not relatively free to mount and support is measured, an operation can be performed conveniently outside the device, thereby reducing uniformity errors among measurers.

### BRIEF DESCRIPTION OF DRAWINGS

FIG. 1 is a diagram of a device for measuring an outdoor air penetration rate of a substrate according to one embodiment of the present invention.

FIG. 2 is a view of a reactant cartridge for measuring the outdoor air penetration rate of the substrate according to one embodiment of the present invention.

FIG. 3 is a view illustrating an example of detaching a shielding means of the reactant cartridge for measuring the outdoor air penetration rate of the substrate of FIG. 2.

FIG. 4 is a view of the reactant cartridge for measuring the outdoor air penetration rate of the substrate according to one embodiment of the present invention.

FIG. 5 is a view illustrating a reaction portion of the reactant cartridge for measuring the outdoor air penetration rate of the substrate of FIG. 4.

FIG. 6 is a view illustrating an example of mounting a substrate mounting unit of the substrate according to one embodiment of the present invention.

FIG. 7 is a view of a substrate cartridge for measuring the outdoor air penetration rate of the substrate according to one embodiment of the present invention.

### DETAILED DESCRIPTION OF EMBODIMENT

Following contents merely illustrate a principle of the present invention. Accordingly, even though not being clearly described or shown in the specification, the principle of the present invention may be embodied and various apparatuses included in the concept and scope of the present invention may be made by one of ordinary skill in the art. Also, all conditional terms and embodiments enumerated herein are clearly intended only to allow the concept of the present invention to be understood but not limited to the embodiments and states particularly enumerated as follows.

Hereinafter, exemplary embodiments of the present invention will be described in detail with reference to the attached drawings.

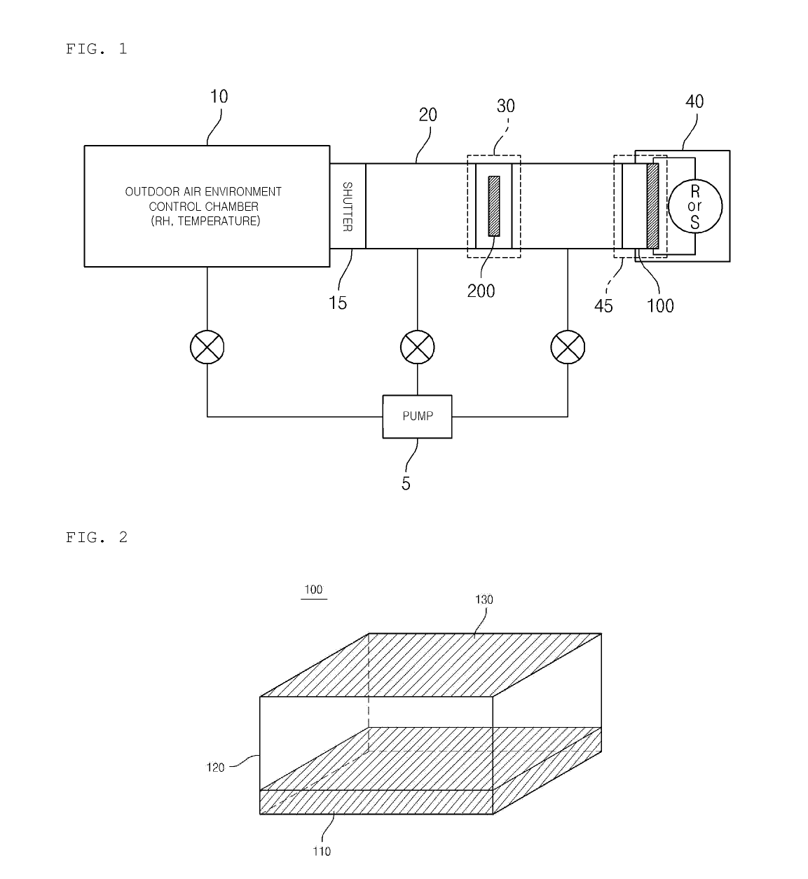

FIG. 1 is a diagram of a device for measuring an outdoor air penetration rate of a substrate according to one embodiment of the present invention.

Referring to FIG. 1, the device for measuring the outdoor air penetration rate of the substrate according to the embodiment includes an outdoor air environment control chamber 10, a supply pipe 20, a substrate mounting portion 30, and a measuring unit 40.

The outdoor air environment control chamber 10 is a component for forming an environment to measure the penetration rate of a substrate 200 and may set current humidity in comparison with to saturable and absorbable humidity at respective temperatures as relative humidity (RH) in the embodiment. The outdoor air environment control chamber 10 supplies outdoor air which includes humidity according to a set environment.

Also, the device for measuring the outdoor air penetration rate of the substrate may further include a shutter 15 on a connection portion between the outdoor air environment control chamber 10 and the supply pipe 20. That is, the shutter 15 is opened to supply the outdoor air according to the controlled environment to the supply pipe 20.

In the embodiment, the supply pipe 20 is formed between the outdoor air environment control chamber 10 and the measuring unit 40 and functions as a path to allow the outdoor air to flow from the outdoor air environment control chamber 10 to the measuring unit 40.

An inside of the supply pipe 20 is formed as a predetermined low pressure state, and preferably, may be formed as a vacuum state or be filled with a gas with small chemical activity such as an inert gas by a pump 5 to increase accuracy of a value measured by the device for measuring the penetration rate.

The substrate mounting portion 30 is located in a first predetermined space of the supply pipe 20, and the substrate 200 to be measured with a penetration rate is mounted thereon. In the embodiment, the substrate 200 is a sample to be measured with an outdoor air penetration rate and may be a plastic substrate 200 of a display panel to be measured with a water vapor transmission rate (WVTR) or a humidity penetration rate. That is, the plastic substrate 200 of the display panel may include a protection layer not to allow humidity to penetrate into the panel to provide reliability of the panel. In the embodiment, the device for measuring the outdoor air penetration rate of the substrate 200 measures a WVTR of the substrate 200 including the protection layer.

In addition, in the embodiment, the first predetermined space is a space between the outdoor air environment control chamber 10 and the measuring unit 40 which will be described below, and the substrate 200 may be formed to allow a wide side of the substrate 200 to be in a direction orthogonal to a flow of the outdoor air in the supply pipe 20 between the outdoor air environment control chamber 10 and the measuring unit 40. That is, the outdoor air which flows through the supply pipe 20 becomes in contact with the substrate 200 in the first predetermined space.

After that, the outdoor air in contact with the substrate 200 penetrates into and is transmitted through the substrate 200 to be in contact with a reactant which will be described below through the supply pipe 20. In the embodiment, the reactant is included in a reactant cartridge 100 and the reactant cartridge 100 is mounted on a cartridge mounting portion 45 located in a second predetermined space of the supply pipe 20. In the embodiment, the second predetermined space is a terminal of the supply pipe 20 and may be a connection portion which connects the supply pipe 20 with the measuring unit 40. Accordingly, the cartridge mounting portion 45 may be included in the measuring unit 40 which measures a change according to a contact with the outdoor air.

As described above, according to a conventional technology, to measure an outdoor air penetration rate of a substrate, a reactant was deposited on the substrate and the penetration rate of the substrate was reversely traced using a change of the reactant. However, to deposit the reactant on the substrate, an additional vacuum deposition apparatus was necessary.

Also, in addition thereto, since a process apparatus for forming a glove box and a sealant necessary for encapsulating a rear portion is necessary, a large amount of time and effort are necessary for analysis. Moreover, since many process parameters occur in analyzing a WVTR depending on a method of depositing a reactant, a structure of a test sample, and a process method, as a result, it is difficult to provide reliable data.

Accordingly, in the embodiment, instead of depositing the reactant on the substrate, the reactant is formed in a cartridge to be detachable from the device for measuring the outdoor air penetration rate in such a way that precise and reliable data compared with the conventional technology may be provided to easily perform reliability evaluation necessary for a new application field such as flexible active matrix organic light emitting diodes (AMOLED).

In the embodiment, the measuring unit 40 measures a change of the reactant according to the contact with the outdoor air.

That is, the measuring unit 40 directly measures the change of the reactant in the reactant cartridge 100 mounted on the cartridge mounting portion 45 at the terminal of the supply pipe 20.

In addition, the pump 5 described above uniformly sets environments of a space between the outdoor air environment control chamber 10 and the first predetermined space and a space between the first predetermined space and the second predetermined space in the supply pipe 20. That is, a low pressure state with the same pressure, and more preferably, a vacuum state or a state of being filled with an inert gas with the same density is preferable.

Hereinafter, referring to FIG. 2, the reactant cartridge 100 according to the embodiment will be described in detail.

FIG. 2 is a view of the reactant cartridge 100 for measuring the outside air penetration rate of the substrate according to one embodiment of the present invention.

Referring to FIG. 2, the reactant cartridge 100 for measuring the outdoor air penetration rate of the substrate according to the embodiment includes a reacting portion 110, a cartridge body 120, and a shielding means 130.

A shape of the reactant cartridge 100 may be variously determined. In the embodiment, it is shown as an example that the reactant cartridge 100 has a rectangular parallelepiped shape. In addition, the shape of the reactant cartridge 100 may differ according to a measurement method of the measuring unit 40 or a shape of the cartridge mounting portion 45, and a size thereof maybe determined according to elements such as a diameter of the supply pipe 20 and a size of a substrate to be measured with a penetration rate.

Hereinafter, a detailed configuration will be described. First, the reacting portion 110 of the reactant cartridge 100 includes a reactant which reacts according to a contact with outdoor air which penetrates the substrate. In FIG. 2, it is shown as an example that the reactant is formed of a reactant layer in a cartridge. When the reactant layer is formed, the measuring unit 40 may measure an optical transmission degree according to the contact with the outdoor air of the reactant layer.

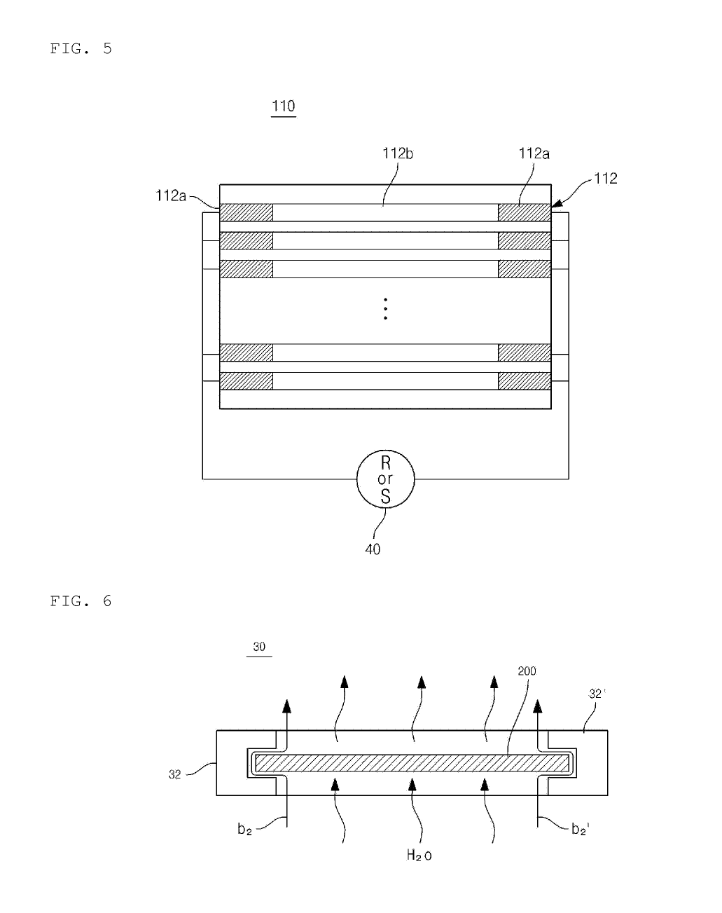

Alternatively, referring to FIG. 4, the reacting portion 110 may be formed as a plurality of reactant bars 112 which consist of a reactant portion 112b which includes a reactant and a conducting portion 112a which includes a conductive material and may be spaced at certain intervals. When the reacting portion 110 is formed of the plurality of reactant bars 112, the measuring unit 40 may measure changes in electrical properties of the reactant bars 112 according to a contact with the outdoor air. In addition, the reactant bars 112 may be formed on a bottom surface of the cartridge body 120 formed of a material such as nonconductive glass.

In more detail, referring to FIG. 5, in the embodiment, both ends of the reactant bars 112 are formed of a conductive material and an intermediate portion is formed of a reactant. The respective conducting portions 112a are electrically connected to the measuring unit 40.

The conducting portions 112a on the ends may be formed of or plated with a material difficult to be oxidized such as Au and Ag. On the contrary, the reactant portion 112b formed of a highly oxidative reactant has a chemical reaction by being in contact with outdoor air including humidity, thereby generating a change in resistance or conductivity.

In addition, in the embodiment, the reactant of the reactant portion 112b may be calcium which quickly reacts with humidity. That is, the measuring unit 40 may measure optical transmittance or resistance variations which will be described below using a calcium layer or a calcium bar.

That is, the device for measuring the outdoor air penetration rate of the substrate according to the embodiment is a device for providing generality of the “calcium test” described above and has an aspect of manufacturing and supplying a calcium layer or a calcium electrode, and components related to measurement of resistance/transmittance which are core but difficult to be independently manufactured in the form of a cartridge.

Through this, a gas penetration rate with a level of 10⁻⁶ g/^{m2}-day may be analyzed and highly reliable data compared with a typical method may be provided.

Referring to FIG. 2 again, the cartridge body 120 accommodates the reacting portion 110 and is formed to be detachable from the measuring unit 40 which measures the change of the reactant according to the contact with the outdoor air. As described above, in the embodiment, it is shown as an example that the reactant cartridge 100 is formed as a rectangular parallelepiped and mounted on the measuring unit 40. Accordingly, the cartridge body 120 may be formed as a rectangular parallelepiped.

In the embodiment, the cartridge body 120, like the mounting of the substrate described above, may be mounted to allow a wide side of the reacting portion 110 to be orthogonal to the flow of the outdoor air in the supply pipe 20.

In addition, the shielding means 130 shields the cartridge body 120 and is detached from the cartridge body 120 to allow the reactant to be in contact with the outdoor air. That is, in the embodiment, the reactant cartridge 100 is mounted on the device for measuring the outdoor air penetration rate in a shielded state, environments of spaces on both sides of the first predetermined space in which the substrate is mounted becomes identical to each other by the pump 5, and then the outdoor air environment control chamber 10 sets humidity and a temperature, thereby detaching the shielding means 130 from the cartridge body 120. In addition, the cartridge body 120 may be a vacuum state which satisfies a predetermined low pressure or may be filled with an inert gas.

The shielding means 130 may be formed of glass in such a way that the detachment thereof may be performed by detaching from the cartridge body 120 or removing like opening a can lid.

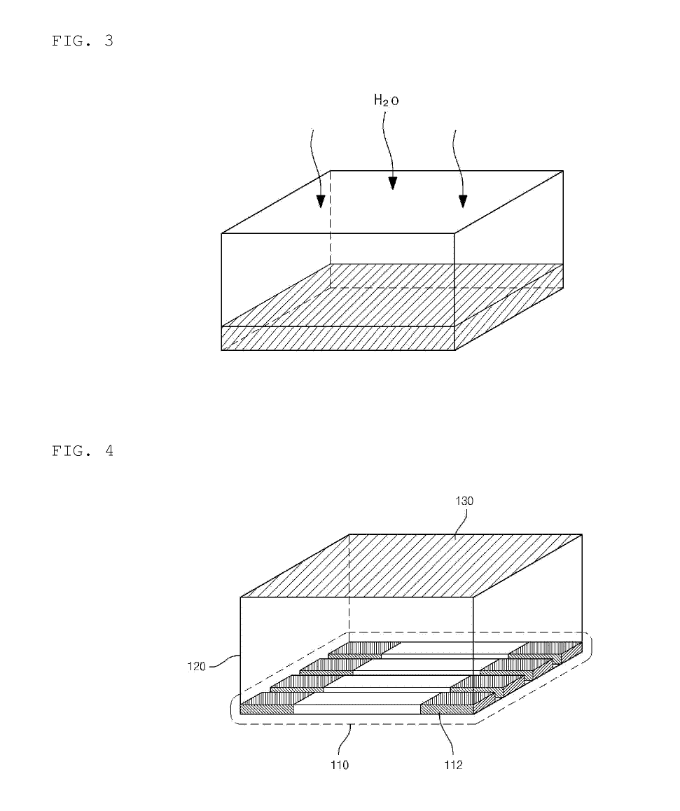

Referring to FIG. 3, when the shielding means 130 of FIG. 2 is detached, the outdoor air including humidity may penetrate into the cartridge and is to be in contact with the reacting portion 110.

Hereinafter, a method of measuring a change of a reactant according to a contact between outdoor air including humidity and the reacting portion 110 will be described.

In the embodiment, the measuring unit 40 measures transmittance of the outdoor air into the reactant layer. That is, an optical transmittance measuring method of measuring transmittance or a resistance method may be used for the measurement.

In the case of the optical transmittance measuring method, the measuring unit 40 measures a change in transmittance of the reactant layer using a photo sensor or an image sensor. In addition, in the resistance method, resistance variations or conductivity variations are analyzed in multipoint analysis using the plurality of reactant bars 112.

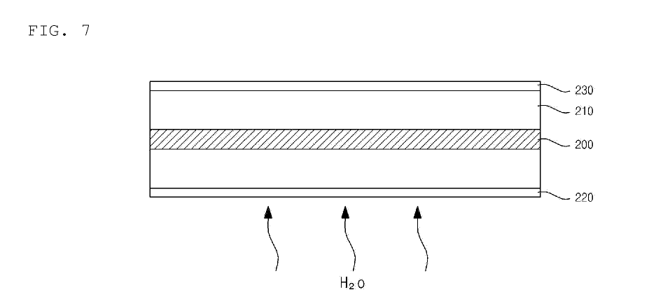

Hereinafter, referring to FIGS. 6 and 7, a substrate cartridge according to one embodiment of the present invention will be described.

FIG. 6 is a view illustrating an example of mounting the substrate 200 on the substrate mounting portion 30. Referring to FIG. 6, when the substrate 200 is mounted on supporting portions 32 and 32′ formed on the substrate mounting portion 30, outdoor air may pass through spaces between the substrate 200 and the supporting portions 32 and 32′ (62 and 62′) without penetration. Since the substrate 200 is not free from mounting and supporting of the substrate mounting portion 30 compared with a panel which is not flexible, such phenomenon may more occur as a display panel becomes more flexible.

Since measurement of an outdoor air penetration rate of the substrate 200 using a reactant considers only an effect caused by outdoor air which penetrates the substrate 200, when outdoor air which passes through the substrate 200 without penetrating into the substrate 200 is in contact with the reactant, an error occurs in the measurement of the outdoor air penetration rate of the substrate 200.

That is, to prevent this case, when the substrate 200 is also manufactured in a cartridge form which accommodates the substrate 200, since it is simply workable outside the device particularly when to measure a flexible substrate which is not free in mounting and supporting, a uniformity error between measures may be reduced.

Referring to FIG. 7, a substrate cartridge which accommodates the substrate according to one embodiment of the present invention, similarly to the reactant cartridge 100 described above, includes a substrate cartridge body 210, the substrate 200, and substrate shielding means 220 and 230.

That is, the substrate cartridge body 210 may be configured to accommodate the substrate 200 and may be detachable from the supply pipe 20. In the embodiment, the substrate cartridge body 210 may be mounted on the substrate mounting portion 30 formed in the first predetermined space.

The substrate shielding means 220 and 230 shield the substrate cartridge body 210 and are detached from the substrate cartridge body 210 to allow the substrate 200 to be in contact with the outdoor air or to allow the reactant to be in contact with the outdoor air which penetrates through the substrate 200. However, the substrate shielding means 220 and 230, unlike the reactant cartridge 100, may be formed on both sides which are a surface at which outdoor air supplied from the environment control chamber 10 is in contact with the substrate 200 and a surface through which outdoor air which penetrates through the substrate 200 is discharged.

As described above, in the embodiment, when environments of spaces on both sides of the first predetermined space in which the substrate 200 is mounted become identical to each other due to the pump 5 and then the outdoor air environment control chamber 10 sets humidity and a temperature, the substrate shielding means 220 and 230 are to be detached from the substrate cartridge body 210.

Hereinafter, a method of measuring a penetration rate of a substrate using the device for measuring the outdoor air penetration rate of the substrate, which includes the reactant cartridge 100, will be described.

First, the reactant cartridge 100 and the substrate cartridge are mounted on the device for measuring the outdoor air penetration rate of the substrate 200 and then a low pressure state, more preferably, a vacuum state or a state of being filled with an inert gas (or dry N₂ atmosphere) is formed. The state herein is formed to allow the spaces on both sides of the supply pipe 20, which are divided by the substrate cartridge, to be in the same environment while the shutter 15 of the outdoor air environment control chamber 10 is closed.

After that, humidity and temperature environments inside the outdoor air environment control chamber 10 are set and the substrate shielding means 220 and 230 of the substrate cartridge and the shielding means 130 of the reactant cartridge 100 are detached.

Next, the shutter 15 is opened and outdoor air including humidity is supplied through the supply pipe 20.

The outdoor air which penetrates into and penetrates through the substrate 200 is in contact with the reactant of the reactant cartridge 100 and reacts therewith.

The measuring unit 40 analyzes resistance variations of the reactant when using the resistance method. That is, an average of the resistance variations of the plurality of reactant bars 112 is extracted. Alternatively, when the optical transmittance method is used, transmittance variations of the reactant layer are sensed and analyzed by a photo sensor or an image sensor.

The above description merely exemplifies the technical concept of the present invention and may be variously modified, changed, and replaced by one of ordinary skill in the art without departing from the technical features of the present invention.

Therefore, the embodiments disclosed herein and attached drawings should not limit the technical concept of the present invention but are only to describe. The scope of the technical concept of the present invention is not limited by the embodiments and the attached drawings but will be interpreted according to the following claims. All equivalents included therein should be understood as being included in the scope of the present invention.

### DESCRIPTION OF REFERENCE NUMERALS

10: Outdoor air environment control chamber
20: Supply pipe
30: Substrate mounting portion
40: Measuring unit
45: Cartridge mounting portion
100: Reactant cartridge
110: Reacting portion
120: Cartridge body
130: Shielding means
200: Substrate
210: Substrate cartridge body
220: Substrate shielding means

What is claimed is:

1. A reactant cartridge for measuring an outdoor air penetration rate of a substrate, comprising:
a reacting portion which comprises a reactant which reacts according to a contact with outdoor air which penetrates through the substrate;
a cartridge body which accommodates the reacting portion and is detachable from a measuring unit which measures a change of the reactant according to the contact with the outdoor air; and
a shielding means which shields the cartridge body and is detached from the cartridge body to allow the reactant to be in contact with the outdoor air.

2. The reactant cartridge of claim 1, wherein the outdoor air in contact with the reactant is outdoor air which comprises humidity penetrating through the substrate.

3. The reactant cartridge of claim 1, wherein the reacting portion is formed of a reactant layer which comprises the reactant, and
wherein the measuring unit measures transmittance of the outdoor air into the reactant layer.

4. The reactant cartridge of claim 1, wherein the reacting portion is formed as a plurality of reactant bars formed of a reactant portion which comprises the reactant and a conducting portion which comprises a conductive material and spaced at certain intervals, and
wherein the measuring unit measures changes in electric properties of the reactant bars.

5. The reactant cartridge of claim 3, wherein the reactant layer is a calcium layer which comprises calcium, and
wherein the measuring unit measures transmittance of the calcium layer.

6. The reactant cartridge of claim 4, wherein the reactant bar is a calcium bar formed of a calcium portion which comprises calcium and the conducting portion, and
wherein the measuring unit measures conductivity or resistance variations of the calcium bar.

7. The reactant cartridge of claim 1, wherein the substrate is accommodated in a substrate cartridge which comprises:
a substrate cartridge body detachable from a supply pipe through which the outdoor air is supplied; and
substrate shielding means which shield the substrate cartridge body and are detached from the substrate cartridge body to allow the substrate to be in contact with the outdoor air or to allow the reactant to be in contact with the outdoor air which penetrates through the substrate.

8. A device for measuring an outdoor air penetration rate of a substrate, which comprises a measuring unit comprising:
an outdoor air environment control chamber which controls an environment of supplied outdoor air;
a supply pipe which supplies the outdoor air controlled with the environment;
a substrate mounting portion located in a first predetermined space of the supply pipe, on which the substrate to be measured with a penetration rate is mounted; and
a cartridge mounting portion located in a second predetermined space of the supply pipe, on which a reactant cartridge which comprises a reactant which reacts according to a contact with outdoor air which penetrates through the substrate is mounted,
wherein the measuring unit measures a change of the reactant according to the contact with the outdoor air.

9. The device of claim 8, wherein the reactant cartridge comprises:
a reacting portion which comprises the reactant which reacts according to the contact with the outdoor air which penetrates through the substrate;
a cartridge body which accommodates the reacting portion and is detachable from the measuring unit which measures the change of the reactant according to the contact with the outdoor air; and
a shielding means which shields the cartridge body and is detached from the cartridge body to allow the reactant to be in contact with the outdoor air.
